(19) United States
(12) Patent Application Publication
(10) Pub. No.: US 2024/0216294 A1
(43) Pub. Date: Jul. 4, 2024

(54) NOVEL USE

(71) Applicant: DSM IP ASSETS B.V., Heerlen (NL)

(72) Inventors: Maik KINDERMANN, Kaiseraugst (CH); Ulla LETINOIS, Kaiseraugst (CH); Luis Fernando MONTEIRO TAMASSIA, Kaiseraugst (CH); René Tobias STEMMLER, Kaiseraugst (CH); Nicola WALKER, Kaiseraugst (CH)

(21) Appl. No.: 18/554,922

(22) PCT Filed: Apr. 12, 2022
(86) PCT No.: PCT/EP2022/059712

§ 371 Date: Oct. 11, 2023

(30) Foreign Application Priority Data
Apr. 15, 2021 (EP) 21168701.7
Jun. 16, 2021 (EP) 21179750.1

(51) Int. Cl. A61K 31/047; A23K 20/105; A23K 20/111; A23K 50/10; A61K 31/05; A61K 31/11; A61K 31/7024; A61P 1/14

(52) U.S. Cl. CPC A61K 31/047; A23K 20/105; A23K 20/111; A23K 50/10; A61K 31/05; A61K 31/11; A61K 31/7024; A61P 1/14

(57) ABSTRACT

The present invention relates to the use of propanediol mononitrate in combination with certain natural substances for reducing the production of methane emanating from the digestive activities of said ruminant as well as in increasing the organic matter digestibility in said ruminant.

The present invention relates to the use of propanediol mononitrate in combination with certain natural phenolic substances for reducing the production of methane emanating from the digestive activities of said ruminant as well as in increasing the organic matter digestibility in said ruminant.

The present invention further relates to animal feed compositions and feed additives comprising propanediol mononitrate and certain natural substances as well as the use of said feed compositions or feed additives for reducing the production of methane emanating from the digestive activities of ruminants and/or increasing the digestibility of organic matter in ruminants.

The temperature of the air surrounding the earth is increasing, a process referred to as global warming. One of the main focuses to reduce this warming effect is to reduce the amount of greenhouse gases emitted into the atmosphere. Greenhouse gases are emitted from several different sources, both natural and artificial; however, the two sources with the most emphasis are the agricultural and fossil fuel industries. Within agriculture, ruminants and in particular cattle are the major contributors to the biogenic methane formation, and it has been estimated that the prevention of methane formation from ruminants would almost stabilize atmospheric methane concentrations.

Methane emission from the ruminant livestock sector—a by-product from enteric fermentation of plant biomass in the ruminant digestive system—is produced by methanogenic archaea. Various attempts have been made in the last decade to mitigate methane production from ruminant animals. Although the approaches vary, the most popular method so far are feed additives which act in the rumen fluid by reducing respectively inhibiting the methane production by methanogenic archaea.

Digestibility is an important factor in the measure of nutrition value of animal feed. Digestibility determines the relation between contents of nutrients and energy that are available to ruminants and can furthermore be used to estimate the protein microbial synthesis in the rumen. Thus, improving organic matter digestibility (OMD) of feed is highly desirable by farmers.

Propanediol mononitrate (also referred to herein as PDMN or 3-Nitrooxypropanol (3-NOP)) having the chemical structure of formula (I) has been reported to significantly reduce the methane production in ruminants (see e.g. WO2012/084629).

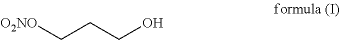

It is an object of the present invention to provide an improved concept to reduce methane emissions from ruminants while preferably also improving the organic matter digestibility.

It has now surprisingly been found, that the combined use of propanediol mononitrate and certain (naturally occurring), preferably phenolic substances leads to a synergistically reduced methane formation while synergistically increasing the organic matter digestibility in ruminants.

Thus the combined use of a propanediol mononitrate and said natural, preferably phenolic substances has a great potential in the mitigation of climate change by significantly reducing the methane emissions emanating during the digestive activities of ruminants while even improving the organic matter digestibility.

Therefore, in a first embodiment, the present invention provides the use of propanediol mononitrate and a mixture of at least six phenolic substances for reducing the formation of methane emanating from the digestive activities of ruminants and/or for improving the organic matter digestibility in ruminants.

In a second embodiment, the invention further provides a method for reducing the production of methane emanating from the digestive activities of ruminants and/or for improving organic matter digestibility in ruminants, said method comprising orally administering to the animal an effective amount of propanediol mononitrate and the mixture of the at least six phenolic substances and optionally appreciating the effect.

In a third embodiment, the present invention relates to a (ruminant) feed composition or feed additive comprising propanediol mononitrate and a mixture of at least six phenolic substances.

In a further embodiment of the present invention, the invention relates to the use of propanediol mononitrate and one or more natural substances selected from the group consisting of 3-hydroxyphenol (resorcinol), tannic acid, 2-methyl-5-(1-methylethyl)-phenol (carvacrol), linalool, anethol (preferably trans anethol), 1-methyl-4-(1-methylethenyl)-cyclohexene (limonene) and 2-hydroxybenzoates (salicylates), preferably benzyl salicylate and isoamyl salicylate for reducing the formation of methane emanating from the digestive activities of ruminants and/or for improving the organic matter digestibility in ruminants.

In an additional embodiment, the invention further provides a method for reducing the production of methane emanating from the digestive activities of ruminants and/or for improving organic matter digestibility in ruminants, said method comprising orally administering to the animal an effective amount of propanediol mononitrate and one or more natural substances selected from the group consisting of 3-hydroxyphenol (resorcinol), tannic acid, 2-methyl-5-(1-methylethyl)-phenol (carvacrol), linalool, anethol (preferably trans anethol), 1-methyl-4-(1-methylethenyl)-cyclohexene (limonene) and 2-hydroxybenzoates (salicylates), preferably benzyl salicylate and isoamyl salicylate and optionally appreciating the effect.

In another embodiment, the present invention relates to a (ruminant) feed composition or feed additive comprising propanediol mononitrate and one or more natural substances selected from the group consisting of 3-hydroxyphenol (resorcinol), tannic acid, 2-methyl-5-(1-methylethyl)-phenol (carvacrol), linalool, anethol (preferably trans anethol), 1-methyl-4-(1-methylethenyl)-cyclohexene (limonene) and 2-hydroxybenzoates (salicylates), preferably benzyl salicylate and isoamyl salicylate.

The term organic matter digestibility as used herein is defined as the proportion of organic matter in the feed that is digested in the ruminant digestive (total) tract. It is generally calculated as percentage of digested (total tract) organic matter to total organic matter in the feed, i.e. in the dry matter (DM).

The phenolic substances according to the invention are a well-known group of compounds found in nature (i.e. naturally occurring phenolic compounds) which are characterized by their structures comprising at least one phenol unit.

Particularly preferred phenolic substances according to the present invention are compounds of formula (I)

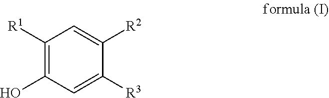

wherein
R¹ is selected from the group consisting of H, C₁₋₅alkyl, C₁₋₅alkoxy and —C(═O)OR⁴, preferably from H, methyl, isopropyl, isoamyl, methoxy, —C(═O)Obenzyl, C(═O)O(3-methyl-2-butanyl) and —C(═O)Oisoamyl;
R² is selected from the group consisting of H, C₁₋₅alkyl, C₂₋₅alkenyl and —C(═O)H, preferably from H, methyl, allyl and —COH;
R³ is selected from the group consisting of H, OH, C₁₋₅alkyl, C₂₋₅alkenyl and C₁₋₅alkoxy, preferably from H, OH, methyl, methoxy, isoamyl, isopropyl and allyl;
R⁴ is selected from the group consisting of C₁₋₅alkyl and C₁₋₅alkylphenyl, preferably from benzyl, 3-methyl-2-butanyl and isoamyl;
as well as tannic acid.

Most preferred compounds of formula (I) in all embodiments of the present invention are the ones wherein
R¹ is selected from the group consisting of H, methyl, isopropyl, methoxy, —C(═O)Obenzyl, C(═O)O(3-methyl-2-butanyl) and —C(═O)Oisoamyl;
R² is selected from the group consisting of H, methyl, allyl and —COH;
R³ is selected from the group consisting of H, OH, methyl, and isopropyl.

The term ‘C₁₋₅alkyl’ as used herein refers to monovalent straight chain or branched, saturated, acyclic hydrocarbyl groups having up to 5 carbon atoms. Preferred alkyl residues in all embodiments of the present invention are methyl, ethyl, n-propyl, i-propyl (isoamyl) or i-, n-, secondary or t-butyl groups,

The term ‘C₂₋₅alkenyl’ as used herein refers to monovalent straight or branched alkyl chains having from 2 to 5 carbon atoms and which have at least one carbon-carbon double bond, which may be (independently from each other) in (E) or (Z)-configuration. Preferably, in all embodiments of the present invention, the C₂₋₅alkenyl group is selected from the group of linear (straight chain) alkenyl groups, such as in particular linear C₂₋₄alkenyl groups, most preferably the C₂₋₅alkenyl group is allyl.

The term ‘alkoxy’ is used herein to refer an —O-alkyl group with all the definitions for alkyl as given above, such as in particular methoxy (—OMe) or ethoxy (—OEt), most preferably methoxy.

The term ‘C₁₋₅alkylphenyl’ is used herein to refer to a —(CH₂)₁₋₅phenyl residue such as preferably benzyl (—(CH₂)phenyl).

It is furthermore preferred, in all embodiments of the present invention at least one of R¹, R² and R³ is not H.

Most preferably, in all embodiments of the present invention, the phenolic substances are selected from the group consisting of 3-hydroxyphenol (CAS No.: 108-46-3), 2-methoxyphenol (CAS No.: 90-05-1), 3-methylphenol (CAS No.: 108-39-4), 2-(1-methylethyl)-5-methylphenol (CAS No.: 89-83-8), 4-allyl-2-methoxyphenol (CAS No.: 97-53-0), 4-hydroxy-3-methoxybenzaldehyd (CAS No.: 121-33-5), 5-isopropyl-2-methylphenol (CAS No.: 499-75-2), 2-hydroxybenzoates (salicylates) and tannic acid (CAS No.: 1401-55-4). Preferred salicylates in all embodiments of the present invention are methyl, ethyl, 3-methyl-2-butanyl, isoamyl and benzyl salicylates. Most preferred in all embodiments of the present invention is the use of benzyl salicylate (CAS No.: 118-58-1), 3-methyl-2-butanyl and/or isoamyl salicylate (CAS No.: 87-20-7), even more preferably benzyl salicylate and/or isoamyl salicylate such as in particular a mixture of both.

Most preferred in all embodiments of the present invention is the use of propanediol mononitrate together with a mixture of at least six, preferably all phenolic substances selected from the group consisting of 3-hydroxyphenol, 2-methoxyphenol, 3-methylphenol, 2-(1-methylethyl)-5-methyl-phenol, 4-allyl-2-methoxyphenol, 4-hydroxy-3-methoxybenzaldehyd, tannic acid and at least one 2-hydroxybenzoates (salicylates) such as preferably benzyl and/or isoamyl salicylate, most preferably benzyl and isoamyl salicylate.

The natural, preferably phenolic substances according to the invention are commercially available or can be prepared by a skilled person using processes and methods well-known in the prior art.

The natural, preferably phenolic substances can be used in highly purified forms or in the form of (e.g. comprised in) natural available plant extracts or extract-mixtures.

The term ‘extract’ as used herein includes compositions obtained by solvent extraction (which are also known as ‘extracted oils’), steam distillation or other methods known to the skilled person. Suitable extraction solvents include alcohols such as ethanol or supercritical carbon dioxide.

The term ‘natural’ in this context of the present invention is to be understood to refer to a substance or a composition (mixture) of substances which substances are occurring in nature and which are obtained from natural products or through synthesis. A particular suitable natural composition for the use in all embodiments of the present invention contains at least six, preferably all of the phenolic substances with all the definitions and preferences as given above as main ingredient and optionally additionally other natural (fragrance) compounds as for example capsaicin or limonene.

It is well understood, that propanediol mononitrate and the one or more natural substances, preferably the mixture of the at least six phenolic substances may be administered admixed together (i.e. premixed) or separately to the ruminant, while in the latter case it is well understood that the administration of propanediol mononitrate and the natural, preferably phenolic substances (separately or, preferably, admixed) occurs within a limited time window, i.e. within at most 6 h, preferably within 3 h, more preferably within 1 h, such as within 0.5 h. Most preferably, if administered separately, all ingredients are concomitantly administered to the ruminant e.g. by concomitant addition thereof into the animal's feed/diet or into the feed rack.

Preferably, in all embodiments of the present invention propanediol mononitrate and the one or more natural substances, such as preferably the mixture of the at least six phenolic substances are administered together in form of a feed composition or feed additive comprising propanediol mononitrate said mixture.

In all embodiments of the present invention it is particularly preferred if propanediol mononitrate and the one or more natural substances such as preferably the mixture of the at least six phenolic substances according to the present invention are administered in the absence of (i.e. not concomitantly with) any antibiotic such a preferably monensin, one or more p-nitroaniline derivative such as p-nitroaniline and/or one or more fatty acid derivative one fatty acid containing at least 5 carbon atoms such as lauric acid.

In one preferred embodiment of the invention propanediol mononitrate and the one or more natural substances such as preferably the mixture of the at least six phenolic substances according to the present invention are added to the feed as single (not premixed) ingredients.

In the uses and methods according to the invention the propanediol mononitrate and the one or more natural substances such as preferably the mixture of the phenolic substances can be fed to the animal before, after, or simultaneously with the diet of the animal. The latter is preferred.

It is well understood, that in all embodiments of the present invention propanediol mononitrate and the one or more natural substances such as preferably the mixture of the at least six phenolic substances are administered to the ruminant in an effective amount, i.e. in an amount which results in a methane reduction, preferably of at least 10%, more preferably of at least 20%, most preferably of at least 30%, such as even of at least 40%, when compared to control, i.e. to ruminants not supplemented with propanediol mononitrate and the mixture of the phenolic substances.

Thus, the present invention also relates to the (combined) use of propanediol mononitrate and the one or more natural substances such as preferably the mixture of the at least six phenolic substances according to the present invention, wherein the methane production in ruminants is reduced by at least 10% compared to control (i.e. in the absence of the phenolic substances and propanediol mononitrate). Preferably, the methane reduction is at least 15%, more preferably at least 20%, even more preferably at least 25%, most preferably at least 30% such as even at least 40%.

The term ‘an effective amount’ as used herein refers to an amount necessary to obtain a reduction of the methane emissions resulting from the digestive activities of a ruminant and/or to increase the organic matter digestibility in a ruminant. It is well understood, that the respective effects may be achieved by one single (daily) dose or by repeated (daily) doses. Furthermore, it is well understood by a person skilled in the art, that the effective amount of the phenolic substances and propanediol mononitrate in the uses, methods and compositions according to the invention may vary depending upon known factors, such as the characteristics of the particular composition and its mode and route of administration, the age, health and weight of the ruminant, the frequency of treatment, all of which can be determined by the expert in the field with normal trials or with the usual considerations regarding the intake regime and/or the formulation.

Propanediol mononitrate [CAS No: 100502-66-7] is a known compound which can e.g. be manufactured as outlined in WO2004043898 or WO2012084629 and which is available under the trademark Bovaer© at DSM Nutritional Products Ltd.

Preferably, in all embodiments of the present invention, the effective amount of propanediol mononitrate to be administered to the ruminants is selected in the range from 0.05 to 5 g PDMN/animal/day, more preferably in the range from 0.1 to 4 g PDMN/animal/day, most preferably in the range from 0.25 to 3 g PDMN/animal/day. Further suitable effective amounts are selected in the range from 0.5 to 3 g PDMN/animal/day, from 1 to 3 g PDMN/animal/day or from 1 to 1.5 g PDMN/animal/day.

With regard to the feed preferably, in all embodiments of the present invention, the effective amount of propanediol mononitrate in the feed administered to the animal per day is selected in the range from 10 mg to 300 mg PDMN/kg DM/day, more preferably in the range from 50 to 150 g PDMN/kg DM/day, most preferably in the range from 60 to 100 g PDMN/kg DM/day.

In all embodiments of the present invention, the propanediol mononitrate is preferably administered e.g. by addition to the feed in the form of a powderous formulation thereof.

Preferably, said powderous formulation is a powderous formulation comprising PDMN and a carrier material. Suitable carrier includes any carrier well known in the food and feed industry such as silicone dioxide (silica) without being limited thereto.

Powderous formulations comprising PDMN and a carrier material are usually prepared by PDMN being sprayed onto or admixed with the carrier material by standard methods in the art, e.g. by diluting PDMN in an organic solvent suitable for the preparation of food or feed products such as e.g. dichloromethane, spraying or admixing said solution with/onto the carrier followed by evaporation of the organic solvent.

Alternatively, PDMN can be diluted in a suitable edible oil before being sprayed onto or admixed with the carrier material. In the latter cases the respective edible oil is generally not removed. The powderous formulation may in addition contain usual additives used in the preparation of powderous formulations for feed application.

The amount of PDMN in the powderous formulation according to the present invention is preferably selected in the range of 1 to 20 wt.-%, preferably in the range of 2 to 15 wt.-%, most preferably in the range of 4 to 12 wt.-%, based on the total weight of the composition.

Particularly suitable powderous formulation to be used in all embodiments of the present invention consists phenolic essentially of PDMN, propylene glycol and silica as e.g. outlined in WO2018/149756 and WO2018/149755 and are commercially available as Bovaer© 10 from DSM Nutritional Products Ltd.

Preferably, in all embodiments of the present invention, the effective amount of the one or more natural substances such as preferably the mixture of the phenolic substances according to the present invention (total amount) with all the definitions and preferences as given herein to be administered to the ruminants is selected in the range from 0.05 to 100 g/animal/day, more preferably in the range from 0.1 to 50 g/animal/day, most preferably in the range from 0.02 to 25 g/animal/day, such as in the range from 0.02 to 10 g/animal/day, 0.02 to 5 g/animal/day or 0.02 to 2.5 g/animal/day. Further suitable ranges are 0.5 to 5 g e/animal/day or 0.5 to 2.5 g/animal/day.

With respect to cattle, it is at present contemplated that the mixture of phenolic substances according to the present invention is administered in amounts (total dosage ranges of phenolic substances/per day) of 0.1 to 150 mg/kg body weight per day, preferably 1 to 150 mg/kg body weight per day, more preferably 50 to 150 mg per kg body weight per day, such as 70 mg to 120 mg per kg body weight per day.

In another preferred embodiment of the invention the one or more natural substances such as preferably the mixture of the phenolic substances of the present invention are added to the feed (diet) of the animal in the form of a single feed additive formulation (also referred to herein as natural respectively phenolic additive formulation).

The term feed additive formulation as used herein refers to a (powderous) formulation, which can either be added directly into an animal feed (diet) or (preferably) be used to prepare a feed additive.

Preferably, said natural or phenolic additive formulation comprises the natural or phenolic substances as defined herein (also referred to as a phenolic substance mixture), at least one carrier and optionally an antioxidant.

Preferably the (total) amount of natural or phenolic substances (i.e. the phenolic substance mixture) in the phenolic additive formulations according to the present invention is selected in the range from 30 to 50 wt.-%, preferably 35 to 45 wt.-%.

Suitable antioxidants to be used in the phenolic additive formulation encompass BHT, BHA, tocopherol or derivatives thereof as well as vitamin C or derivatives thereof. The antioxidants are generally used in amounts up to 2 wt.-%, based on the total weight of the phenolic additive formulation.

Suitable carriers to be used in the phenolic additive formulation are well known to a person skilled in the art and encompass silicon dioxide (silica), proteins as well as plant fibres and middlings (organic carriers) without being limited thereto. Preferably, the carrier is a mixture of silica and one or more organic carriers. Said carriers are generally present in amounts of 40 to 60 wt.-%, preferably 45 to 55 wt.-%, based on the total weight of the phenolic additive formulation. It is well understood that the carriers are inert.

Examples of particularly preferred dosages of the individual natural respectively phenolic substances in a natural or phenolic additive formulation according to the present invention are selected in the following ranges:
2-(1-methylethyl)-5-methyl-phenol between 7.5 to 15 wt.-%, preferably 8 to 12 wt.-%;
4-allyl-2-methoxyphenol between 2 and 6 wt.-%, preferably 2.5 to 5 wt.-%;
3-methylphenol 7.5 to 15 wt.-%, preferably 8 to 12 wt.-%;
4-hydroxy-3-methoxybenzaldehyd between 3 and 7 wt.-%, preferably 4 to 6 wt.-%;
2-methoxyphenol between 2 and 5 wt.-%, preferably 2.5 to 4 wt.-%;
2-hydroxybenzoates, preferably a mixture of benzyl salicylate and isoamyl salicylate, between 1 to 5 wt.-%, preferably 2 to 4 wt.-%;
3-hydroxyphenol between 1 to 3 wt.-%, preferably 1 to 2 wt.-%;
tannic acid between 0.5 to 15 wt.-%, preferably 0.75 to 3 wt.-%;
up to about 5 wt.-%, preferably up to 3 wt.-% of limonene, anethol and/or linalool
   with all wt.-% being based on the total weight of the natural or phenolic additive formulation.

It is well understood, that the remaining ingredients in the phenolic additive formulation are the carrier(s) and the optional antioxidant.

It is well understood, however, that the natural or phenolic additive formulations according to the present invention may optionally contain, preferably in minor amounts, other natural (fragrance) substances, preferably selected from the following group, and in amounts as indicated based on the single feed additive composition:
up to about 1 wt.-%, preferably up to 0.5 wt.-% of propylidene- and/or butylidenephtalides, isophorones, gingerol and/or lavender oil;
up to about 2 wt.-%, preferably up to about 1 wt.-% of deca-, undeca- and/or dodecalactones, ionones, irone, eucalyptol, menthol, peppermint oil and/or alpha-pinene;
up to about 5 wt.-%, preferably up to 3 wt.-% of limonene, anethol, linalool and/or methyl dihydrojasmonate;
up to about 4 wt.-%, preferably up to 2 wt.-% of propionic, acetic or butyric acid, rosemary oil, clove oil, geraniol, terpineol and/or citronellol;
up to about 3 wt.-%, preferably up to 2 wt.-% of cinnamaldehyde and/or a plant polyphenol (tannin);
and up to about 5 wt.-%, preferably up to 2 wt.-% of a powder of turmeric and/or of an extract of Curcuma.

All the natural, preferably phenolic substances and the additional compounds may be used in combination with an emulsifying surfactant.

The emulsifying agent can be selected advantageously from those of a rather hydrophilic nature, for example among polyglycerol esters of fatty acids such as esterified ricinoleic acid or propylene glycol esters of fatty acids, saccharo-esters or saccharo-glycerides, polyethylene glycol, lecithins etc.

For purposes of the present invention, a preferred phenolic additive formulation according to the claims is available under the commercial product name Crina® Ruminants (available from DSM Nutritional Products AG, Kaiseraugst, 30 Switzerland). Crina® Ruminants is a blend of flavouring compounds as outlined above adsorbed on a carrier for animal nutrition and has content of about 380-420 g/kg (38-42%) total phenolic (aromatic) substances.

Propanediol mononitrate and the one or more natural substances such as preferably the mixture of the at least six phenolic substances in all uses and methods according to the present invention are preferably administered concomitantly to the ruminant, either by prior admixing thereof or by separate addition to the diet of the animal.

Propanediol mononitrate and the one or more natural substances such as preferably the mixture of the at least six phenolic substances are preferably administered via a (ruminant) feed composition or feed additive to the ruminant, e.g. by admixing the individual components with the ruminant's feed (diet).

Thus, preferably, in all uses and methods according to the present invention propanediol mononitrate as well as the one or more natural substances such as preferably the mixture of the at least six phenolic substances with all the definitions and preferences as given herein are administered to the ruminant incorporated into a (ruminant) feed composition or feed additive (premix).

The term feed composition or feed additive as used herein means any preparation, mixture, or composition suitable for, or intended for oral intake by an animal. Exemplary feed for ruminants such as cows include forage (grass, legumes, silage), hay, grass, grain as well as soy without being limited thereto.

Said feed compositions or feed additives may be prepared by methods known per se in the art of feed formulation and processing.

Said feed compositions and feed additives are still novel. Thus, further aspects of the present invention are (ruminant) feed compositions and feed additives comprising propanediol mononitrate the one or more natural substances such as preferably the mixture of the at least six phenolic substances with all the definitions and preferences as given herein.

In one preferred embodiment, the feed compositions and feed additives are a mineral premix, a vitamin premix including vitamins and optionally minerals or a bolus.

Propanediol mononitrate and the one or more natural substances such as preferably the mixture of the at least six phenolic substances with all the definitions and preferences as given herein may be used in combination with conventional ingredients present in an animal feed composition (diet) such as forages (raw, grass, hay, silages), co-products from industry (citrus pulp, soybean hulls, distillers dried or wet grains with solubles, minerals (calcium carbonates, electrolytes such as ammonium chloride, macro and trace minerals and in all forms, inorganic and organic), proteins such as soya bean meal, sunflower meal, meat and bone meal, fish meal, amino acids and others, energy ingredients such as grains, wheat, starch, barley, millet, sorghum, corn, animal and plants fat or oils, and vitamins without being limited thereto.

Thus, the present invention also relates to uses and methods according to the present invention, wherein propanediol mononitrate and the one or more natural substances such as preferably the mixture of the at least six phenolic substances with all the definitions and preferences as given herein are supplemented together.

Particular examples of feed compositions of the invention are the following:
An animal feed additive comprising (a) propanediol mononitrate and (b) the mixture of the at least six phenolic substances and one or more of (c) fat-soluble vitamin(s), (d) water-soluble vitamin(s), (e) trace mineral(s) and (f) macro mineral(s);
An animal feed composition comprising (a) propanediol mononitrate and (b) the mixture of the at least six phenolic substances and (c) one or more of (c-1) a crude protein content of 50 to 800 g/kg feed (50-80%), (c-2) fat from 5-100 g/kg feed (5-10%), (c-3) NDF from 150-700 g/kg feed (15-70%), (c-4) TDN from 300-800 (30-80%) and (c-5) starch from 150-700 g/kg feed (15-70%).

It is well understood, that all definitions and preferences as given herein also apply to the animal feed additive and animal feed compositions as disclosed herein.

The so-called premixes are examples of animal feed additives of the invention. A premix designates a preferably uniform mixture of one or more micro-ingredients with diluents and/or carrier. Premixes are used to facilitate uniform dispersion of micro-ingredients in a larger mix.

Apart from the active ingredients of the invention (i.e. the propanediol mononitrate and the one or more natural substance such as preferably the mixture of the phenolic substances), the premix of the invention preferably contains at least one fat-soluble vitamin, and/or at least one water soluble vitamin, and/or at least one trace mineral, and/or at least one macro mineral. In other words, the premix of the invention comprises the propanediol mononitrate and the mixture of the at least six phenolic substances together with at least one additional component selected from the group consisting of fat-soluble vitamins, water-soluble vitamins, trace minerals, and macro minerals.

Macro minerals may be separately added to the feed. Therefore, in a particular embodiment, the premix comprises the mixture of the at least six phenolic substances and propanediol mononitrate with at least one additional component selected from the group consisting of fat-soluble vitamins, water-soluble vitamins, and trace-minerals.

The following are non-exclusive lists of examples of these components:
Examples of fat-soluble vitamins are vitamin A, vitamin D3, vitamin E, and vitamin K, e.g. vitamin K3.
Examples of water-soluble vitamins are vitamin B12, biotin and choline, vitamin B1, vitamin B2, vitamin B6, niacin, folic acid and panthothenate, e.g. Ca-D-panthothenate.
Examples of trace minerals are manganese, zinc, iron, copper, iodine, selenium, manganese, and cobalt.
Examples of macro minerals are calcium, phosphorus, potassium, magnesium and sodium.

A premix may comprise 0.5 to 10% by weight of the active ingredients according to the invention (i.e. the propanediol mononitrate and the one or more natural substances such as preferably the mixture of the at least six phenolic substances) and 10 to 95% by weight of other conventional additives, such as flavorings, vitamins, mineral salts and any conventional absorbing support. This premix is finally added to the feed.

As regards feed compositions for ruminants such as cows, as well as ingredients thereof, the ruminant diet is usually composed of an easily degradable fraction (named concentrate) and a fiber-rich less readily degradable fraction (named hay, forage, or roughage).

Hay is made of dried grass, legume or whole cereals. Grasses include among others temperate or tropical grasses, timothy, ryegrasses, fescues, brachiaria, panicum, tifton. Legumes include among others clover, lucerne or alfalfa, peas, beans and vetches. Whole cereals include among others barley, maize (corn), oat, wheat, sorghum. Other forage crops include sugarcane, kales, rapes, and cabbages. Also root crops such as turnips, swedes, mangles, fodder beet, and sugar beet (including sugar beet pulp and beet molasses) are used to feed ruminants. Still further crops are tubers such as potatoes, cassava and sweet potato. Silage is an ensiled version of the fiber-rich fraction (e.g. from grasses, legumes or whole cereals) and grains (e.g. high moisture corn silage) whereby material with a high water content is treated with a controlled anaerobic fermentation process (naturally-fermented or additive treated) without being limited thereto.

Concentrate feed is largely made up of cereals grains (such as barley including brewers grain and distillers grain, maize, wheat, sorghum), but also often contain protein-rich feed ingredients such as soybean meal, rapeseed meal, palm kernel and sunflower meal without being limited thereto.

Ruminants (males, females and in all growth stages and adults) may also be fed total mixed rations (TMR), where all the dietary components, e.g. forage, silage, other feed ingredients and concentrate, are mixed before serving.

As mentioned above a premix is an example of a feed additive which may comprise propanediol mononitrate and the one or more natural substances such as preferably the mixture of the at least six phenolic substances as defined herein and. It is understood that the compounds may be administered to the animal in different other forms. For example the compounds can also be included in a bolus that would be placed in the rumen and that would release a defined amount of the active compounds continuously in well-defined dosages over a specific period of time.

In a particular advantageous embodiment the feed composition according to the present invention is a ruminant feed (often also referred to as ruminant diet) where all the dietary components, e.g. macro and micro ingredients, forage, silage and concentrate feed and additives including the one or more natural substances such as preferably the mixture of the phenolic substances and propanediol mononitrate are included. Such ruminant feed or diet is also often referred to as total mixed ration (TMR) or Partial mixed ration (PMR) or nutritional supplement for grazing animals.

Preferably, in all embodiments, in said ruminant feed the amount of the propanediol mononitrate is selected in the range from 1 mg to about 25 g per kg dry matter feed, preferably from about 1 mg to about 10 g per kg dry matter feed, more preferably from about 10 mg to about 1 g per Kg dry matter feed, most preferably from 20 mg to 500 mg per Kg of dry matter feed, such as from about 20 mg to 250 mg per Kg of dry matter feed, or even more preferably from 10 mg to 300 mg per dry matter kg feed such as in the range from 50 mg to 150 mg per kg dry matter feed or 60 mg to 100 mg per kg dry matter feed.

Preferably, in all embodiments, in said ruminant feed the amount of the one or more natural substances such as preferably the mixture of the at least six phenolic substances is selected in the range from 10 mg to about 500 mg mixture per kg dry matter feed, preferably from about 10 mg to about 250 mg mixture per Kg dry matter feed, more preferably from 10 mg to 100 mg mixture per Kg of dry matter feed. Further particular suitable ranges are from 10 mg to 75 mg mixture per Kg of dry matter feed or 20 mg to 50 mg mixture per Kg of dry matter feed.

It is also well understood that in all uses and methods according to the present invention propanediol mononitrate and the one or more natural substances such as preferably the mixture of the at least six phenolic substances as defined herein have to be supplemented timely together to excerpt the synergistic effect, can however been added separately to the diet of the animal, e.g. can be added separately into the respecting feeding rack.

Thus, the present invention relates to a method of supplementing propanediol mononitrate and the one or more natural substances such as preferably the mixture of the at least six phenolic substances with the preferences and definitions as given herein to a ruminant, said method encompassing the step of adding propanediol mononitrate and the one or more natural substances such as preferably the mixture of the at least six phenolic substances concomitantly to a feed rack.

The amount of ruminant feed administered to a ruminant may vary dependent on the kind and age. Generally the amount of dry matter fed to beef cattle or dairy cows is selected in the range of 1.5% to 3.5% of live weight, such as for an animal of 500 kg of live weigh it means to feed 7.5 to 17.5 kg dry matter/d.

In all embodiments of the present invention, it is to be understood by oral administration, a simple feeding, or manual administration of a bolus. It is also well understood, that propanediol mononitrate and the one or more natural substances such as preferably the mixture of the at least six phenolic substances according to the present invention can be pre-mixed before the administration or can be added separately to the animal feed compositions and feed additives.

Methane emission by ruminants can easily be measured in individual animals in metabolic chambers by methods known in the art (Grainger et al., 2007 J. Dairy Science; 90: 2755-2766). Moreover, it can also be assessed at barn level by an emerging technology using laser beam (McGinn et al., 2009, Journal of Environmental Quality; 38: 1796-1802) or Sulfur hexafluoride or just SF6 or GreenFeed system. Alternatively, methane produced by a dairy ruminant can also be assessed by measurement of fatty acid profiles in milk according to WO 2009/156453.

The present invention also relates to the use of a feed composition or feed additive according to the present invention which feed composition or feed additive comprises one or more additional active substance which shows similar effects with regard to methane formation in the rumen and which is selected from the group consisting of diallyl disulfide, garlic oil, allyl isothiocyanate, deoxycholic acid, chenodeoxycholic acid and derivatives thereof.

Further components that could be given together with propanediol mononitrate and the one or more natural substances such as preferably the mixture of the at least six phenolic substances are for example yeasts.

It is at present contemplated that diallyl disulfide, garlic oil, allyl isothiocyanate deoxycholic acid, chenodeoxycholic acid and derivatives thereof are independently administered in dosage ranges of for example 0.01-500 mg active substance per kg feed (ppm). These compounds are either commercially available or can easily be prepared by a skilled person using processes and methods well-known in the prior art.

Ruminating mammals according to the present invention include cattle, goats, sheep, giraffes, American Bison, European bison, yaks, water buffalo, deer, camels, alpacas, llamas, wildebeest, antelope, pronghorn, and Nilgai.

For all embodiments of the present invention, domestic cattle, sheep and goat are the more preferred species. For the present purposes most preferred species are domestic cattle. The term includes all races of domestic cattle, and all production kinds of cattle, in particular dairy cows and beef cattle. It is well understood, that the term dairy cows and beef cattle encompasses animals in all ages and physiological stage of life and production systems such as confined, semi-confined and grazing.

In further embodiments, the present invention relates to the use of one or more natural substances such as preferably the mixture of the at least six phenolic substances as defined herein to synergistically enhance the methane reducing properties of propanediol mononitrate in ruminants. It is well understood that all the definitions and properties as defined herein also apply to said use.

The present invention is further described by the following examples which should not be construed as limiting the scope of the invention.

### EXAMPLE

The methane production and the effect on digested organic matter were analysed according to Fermentrics Interpretation and Guidelines (2013) in quadruplicate Fermentrics© vessels fed high quality dairy diets (400 mg dry matter (DM); 60% high quality corn silage) at Fermentrics (RFS Technologies, Ottawa, ON).
Tested products: Phenolic additive formulation comprising approx. 40 wt.-% of phenolic substance mixture and approx. 60 wt. % carrier (PAF); Phenolic substance mixture: 4-hydroxy-3-methoxybenzaldehyd (13%), 2-(1-methylethyl)-5-methyl-phenol (27%), 3-methylphenol (27%), 3-hydroxyphenol (4%), 2-methoxyphenol (9%), 4-allyl-2-methoxyphenol (10%), salicylates (8%), tannic acid (4%).
   Carrier: silica/organic
   Propanediol mononitrate (10% on silica) (3-NOP/S)
Tested concentrations: 3-NOP (based on the active): 80 mg/kg DM
   CRINA: 40 mg/kg DM,
   respectively the combination thereof (3-NOP/S & PAF).

The results are presented in the following Table 1. Clear synergistic effects were obtained for the methane reduction when propanediol mononitrate was combined with a phenolic substance formulation according to the present invention as well as for the digestibility of organic matter which translates into additional performance benefits for the animal.

TABLE 1
Effect on Methane production after 24 h

| | | Total methane | Delta | |
|---|---|---|---|---|
| # | Test | [ml CH₄] | [ml CH₄] | Δ* |
| 1 | Control | 13.5 | — | |
| 2 | 3-NOP/S | 11.4 | −2.1 | −15% |
| 3 | PAF | 12.5 | −1 | −7.4% |
| 4 | 3-NOP/S & PAF | 7.8 | −5.7 | −42% |

*based on control

TABLE 2
Effect on organic matter digested at 24 h

| | | Fraction of | | |
|---|---|---|---|---|
| | | initial DM | Delta* | |
| # | Test | [%] | [%] | Δ* |
| 1 | Control | 62.3 | — | |
| 2 | 3-NOP/S | 61.6 | −0.7 | −1% |
| 3 | PAF | 62.4 | +0.1 | +0.2% |
| 4 | 3-NOP/S & PAF | 63.2 | +0.9 | +1.4% |

*based on control

1. Use of propanediol mononitrate (PDMN) and a mixture of at least six phenolic substances for reducing the formation of methane emanating from the digestive activities of ruminants and/or for improving the organic matter digestibility in ruminants.

2. The use according to claim 1, wherein the at least six phenolic substances are selected from the group consisting of tannic acid and compounds of formula (I) 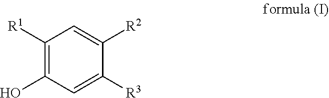
wherein
R¹ is selected from the group consisting of H, C₁₋₅alkyl, C₁₋₅alkoxy and —C(═O)OR⁴, preferably from H, methyl, isopropyl, isoamyl, methoxy, —C(═O)Obenzyl, C(═O)O(3-methyl-2-butanyl) and —C(═O)Oisoamyl;
R² is selected from the group consisting of H, C₁₋₅alkyl, C₂₋₅alkenyl and —C(═O)H, preferably from H, methyl, allyl and —C(═O)H;
R³ is selected from the group consisting of H, OH, C₁₋₅alkyl, C₂₋₅alkenyl and C₁₋₅alkoxy, preferably from H, OH, methyl, methoxy, isoamyl, isopropyl and allyl;
R⁴ is selected from the group consisting of C₁₋₅alkyl and C₁₋₅alkylphenyl, preferably from benzyl, 3-methyl-2-butanyl and isoamyl.

3. The use according to claim 1, wherein the at least six phenolic substances are selected from the group consisting of 3-hydroxyphenol, 2-methoxyphenol, 3-methylphenol, 2-(1-methylethyl)-5-methyl-phenol, 4-allyl-2-methoxyphenol, 4-hydroxy-3-methoxybenzaldehyd, 5-isopropyl-2-methylphenol, 2 hydroxybenzoates (salicylates) and tannic acid.

4. The use according to claim 1, wherein the mixture of phenolic substances comprises at least six, preferably all of 3-hydroxyphenol, 2-methoxyphenol, 3-methylphenol, 2-(1-methylethyl)-5-methyl-phenol, 4-allyl-2-methoxyphenol, 4-hydroxy-3-methoxybenzaldehyd, tannic acid, benzyl salicylate and isoamyl salicylate.

5. The use according to claim 1, wherein propanediol mononitrate is administered to the ruminant in an amount selected in the range from 0.05 to 5 g PDMN/animal/day, preferably from 0.1 to 4 g PDMN/animal/day, most preferably from 0.25 to 3 g PDMN/animal/day.

6. The use according to according to claim 1, wherein the phenolic substances (total amount) are administered to the ruminant in amounts of 0.1 to 150 mg/kg body weight per day, preferably 1 to 150 mg/kg body weight per day, more preferably 50 to 150 mg per kg body weight per day.

7. The use according to claim 1, wherein the ruminant animal is selected from the group consisting of domestic cattle, preferably from beef cattle or dairy cows.

8. The use according to claim 1, wherein the methane production is reduced by at least 10%, preferably by at least 15%, more preferably by at least 20%, most preferably by at least 25% compared to a non-treated control.

9. A method for reducing the production of methane emanating from the digestive activities of ruminants and/or for improving the organic matter digestibility in ruminants, said method comprising orally administering to the ruminant an effective amount of (a) propanediol mononitrate and (b) a mixture of at least six phenolic substances, preferably selected from the group consisting of 3-hydroxyphenol, 2-methoxyphenol, 3-methylphenol, 2-(1-methylethyl)-5-methyl-phenol, 4-allyl-2-methoxyphenol, 4-hydroxy-3-methoxybenzaldehyd, 5-isopropyl-2-methylphenol, tannic acid and 2 hydroxybenzoates (salicylates).

10. The method according to claim 9, wherein the effective amount of propanediol mononitrate is selected in the range from 0.05 to 5 g PDMN/animal/day, preferably from 0.1 to 4 g PDMN/animal/day, most preferably from 0.25 to 3 g PDMN/animal/day.

11. The method according to claim 9, wherein the phenolic substances are administered to the ruminant in an amount (total) of 0.1 to 150 mg/kg body weight per day, preferably 1 to 150 mg/kg body weight per day, more preferably 50 to 150 mg per kg body weight per day.

12. The method according to claim 9, wherein the ruminant animal is selected from the group consisting of domestic cattle, preferably from beef cattle or dairy cows.

13. A feed composition or feed additive comprising propanediol mononitrate and a mixture of at least six phenolic substances, preferably selected from the group consisting of 3-hydroxyphenol, 2-methoxyphenol, 3-methylphenol, 2-(1-methylethyl)-5-methyl-phenol, 4-allyl-2-methoxyphenol, 4-hydroxy-3-methoxybenzaldehyd, 5-isopropyl-2-methylphenol, tannic acid and 2 hydroxybenzoates (salicylates).

14. The feed composition according to claim 13, wherein the feed composition is a ruminant feed comprising the mixture of the phenolic substances in an amount selected in the range from 10 mg to about 100 mg mixture per kg dry matter feed and propanediol mononitrate in an amount selected in the range from 20 mg to 250 mg propanediol mononitrate per kg dry matter feed.

15. Use of a mixture of at least six phenolic substances, preferably selected from the group consisting of 3-hydroxyphenol, 2-methoxyphenol, 3-methylphenol, 2-(1-methylethyl)-5-methyl-phenol, 4-allyl-2-methoxyphenol, 4-hydroxy-3-methoxybenzaldehyd, 5-isopropyl-2-methylphenol, tannic acid and 2 hydroxybenzoates (salicylates), preferably benzyl salicylate and isoamyl salicylate, more preferably selected from the group consisting of 3-hydroxyphenol, 2-methoxyphenol, 3-methylphenol, 2-(1-methylethyl)-5-methyl-phenol, 4-allyl-2-methoxyphenol, 4-hydroxy-3-methoxybenzaldehyd, tannic acid, benzyl salicylate and isoamyl salicylate to enhance synergistically the methane reducing properties of propanediol mononitrate in ruminants and/or the organic matter digestibility in ruminants.
